(19) United States
(12) Patent Application Publication
(10) Pub. No.: US 2015/0250978 A1
(43) Pub. Date: Sep. 10, 2015

(54) Infant Incubator Audio Therapy System

(71) Applicants: Jill Pelsue, St. Louis, MO (US); Grace L. Pelsue, St. Louis, MO (US)

(72) Inventors: Jill Pelsue, St. Louis, MO (US); Grace L. Pelsue, St. Louis, MO (US)

(21) Appl. No.: 14/337,504

(22) Filed: Jul. 22, 2014

Related U.S. Application Data
(60) Provisional application No. 61/947,534, filed on Mar. 4, 2014.

(51) Int. Cl. A61M 21/02; H04R 1/02

(52) U.S. Cl. CPC A61M 21/02; H04R 1/028; A61M 2021/0027

(57) ABSTRACT

Provided is an audio therapy system designed for use with incubated infants. The system includes an audio playback device configured to store and play back recordings of soothing music, a mother's heartbeat, a mother's voice, or nature sounds. This audio playback device electrically and operatively couples to one or more speaker units, which are removably secured to the interior of an incubation unit. In this manner, audio playback from the audio is played within the interior of an incubation unit to calm and soothe an infant contained therein. The audio playback device is stored within a cradle that removably secures to the exterior of an incubation unit, to reduce the likelihood that audio therapy will be abruptly initiated or discontinued.

### CROSS REFERENCE TO RELATED APPLICATION

This application claims the benefit of U.S. Provisional Application No. 61/947,534 filed on Mar. 4, 2014 entitled “Neonate Tranquility.” The above identified patent application is herein incorporated by reference in its entirety to provide continuity of disclosure.

### BACKGROUND OF THE INVENTION

1. Field of the Invention

The present invention relates to an audio therapy system. More specifically, it relates to a portable audio playback system that can be retrofit to an infant incubator. Soothing music, audio recordings of parental voices, or the sound of a heartbeat may be played throughout the incubator to sooth the contained infant.

Infants that are born prematurely, or well in advance of the mother's expected due date, can suffer from a wide variety of health problems. It is common for these babies to have reduced body temperature and difficulty breathing due to the underdeveloped nature of their circulatory and pulmonary systems. These problems, though life threatening, can be addressed through medical assistance in the form of life support systems the enable the infant's body to continue developing. Once the infant's biological systems become self-sustaining, the baby is ready to go home with its family.

Infant life support systems are usually associated with incubation units. Babies in need of constant medical assistance are placed underneath or within one of these domed structures to protect them from the outside world. Air holes and access openings are placed throughout the walls of the incubation unit to facilitate respiration. Some incubation units have heaters that warm the air within the incubator, helping to keep the infant warm until it is able to maintain its own body temperature. Similarly, the incubation unit may be equipped with CPAP machines and other respiration aids.

The problem with incubation units Is that they isolate the infant from its mother during the period when she is most needed. Infants in incubation may become distressed at the separation from their mother, or may have trouble remaining calm without her presence. Because admittance to infant intensive care wards is limited, mothers may have a difficult time being around their children as often as they'd like.

A system is needed to presenting incubated infants with comforting reminders of their mother. The present system provides audio therapy to incubated infants by producing audio playback of a mother's voice, heartbeat, or other soothing sound.

2. Description of the Prior Art

Devices have been disclosed in the prior art that relate to audio playback systems for incubators. These include devices that have been patented and published in patent application publications. These devices generally relate to playing audio inside or near an infant incubator. The following is a list of devices deemed most relevant to the present disclosure, which are herein described for the purposes of highlighting and differentiating the unique aspects of the present invention, and further highlighting the drawbacks existing in the prior art.

Kneuer, U.S. patent application publication no. 2013/0282198 discloses a user interface for controlling functionality of an infant incubator. The device has a graphical user interface display panel and an input means. The device is secured to the exterior of an infant incubator and is electrically connected to the incubator's systems. Temperature, lighting, heart monitors and the like, may be controlled through a single interface. The device is capable of audio playback, which is controlled by caregivers. Soothing sounds may be played at the display panel terminal. This device does not include a modular audio playback device, a mounted playback device holder, or a speaker secured into the wall of the incubator.

McClain, U.S. Pat. No. 6,409,654 discloses an incubator monitoring system with communication capabilities. The monitoring system includes a microphone and speakers to enable bi-directional communication between an incubated infant and a caregiver. Audio playback may include the sound of the mother's voice, or the speech of an attending nurse. The McClain system does not include a portable audio playback device or a holding receptacle mounted to the side of the incubator. The present invention provides a system having a portable audio playback device that can be preprogrammed with a variety of sound options. The device is stored on a receptacle on the side of the incubator and sends audio data directly to a speaker installed in the side of the incubator.

Kolarovic, U.S. Patent Application Publication No. 2002/0173696 discloses an infant incubator with sensor detectors and sensory output means. Sensors may include infrared, cameras, microphones, heartbeat monitors and the like. The speakers may be used to output noise cancellation, such as white noise, into the infant incubator. Unlike the present invention, the system does not include a portable audio output device that can be used to record the sound of a heart beat, voice, or music.

Other types of sound systems for children involve easily accessible structures with integrated sound systems. By way of example, U.S. patent application publication no. 2011/0318721 by Kespy-Yahi, discloses a dome-like structure that has multiple entrances for an independently mobile child. A set of loudspeakers is integrated into the structure and is electrically coupled to a set of controls and an exterior user interface. Sound files can be programmed into the structure via the exterior user interface, i.e. an application on a computer. When the child interacts with the internal control buttons, sounds are played back throughout the enclosure. This system does not include a removable audio playback device, nor does it include a receptacle for storing the audio playback device.

These prior art devices have several known drawbacks. They do not present a modular system that can be moved piecemeal from incubation unit to incubation unit. These systems do not have sterile protective covers for speaker units, nor do they offer storage cradles for retaining a portable audio playback device. The present invention includes these features and additional functionality associated therewith. It substantially diverges in design elements from the prior art and consequently it is clear that there is a need in the art for an improvement to audio therapy systems. In this regard the instant invention substantially fulfills these needs.

### SUMMARY OF THE INVENTION

In view of the foregoing disadvantages inherent in the known types of audio therapy systems now present in the prior art, the present invention provides a new modular components wherein the same can be utilized for providing convenience for the user when utilizing audio therapy across several incubation units.

It is therefore an object of the present invention to provide a new and improved audio therapy system that has all of the advantages of the prior art and none of the disadvantages.

It is another object of the present invention to provide an audio therapy system adapted for use with infant incubation units.

Another object of the present invention is to provide an audio therapy system having modular components that can be easily transferred to other sites.

Yet another object of the present invention is to provide an audio therapy system that includes modular components protected from airborne contaminants and unintentional adjustment.

Still another object of the present invention is to provide an audio therapy system that can sooth incubated infants with the sound of a mother's voice or heartbeat.

A further object of the present invention is to provide an audio therapy system that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the cradle element being attached to the exterior side of an incubator wall. Audio playback device is removably inserted into the cradle for storage during system operation.

FIG. 2 shows a perspective view of a speaker being removably secured to the inside of an incubator wall.

FIG. 3 shows a perspective view of the system during set up. The audio playback device is connected to an audio cable, and placed within the cradle. A speaker removably secured to the interior of the incubator is also connected to the audio cable to enable output of audio data.

FIG. 4 shows front view of a sanitary screen/dust cover being applied to the speaker.

FIG. 5 shows a front view of the speaker with protective screen in place, and an instruction label affixed thereto.

FIG. 6 shows a perspective view of the system in use. Soothing music is output from the speaker, into the interior of the incubator device.

### DETAILED DESCRIPTION OF THE INVENTION

Reference is made herein to the attached drawings. Like reference numerals are used throughout the drawings to depict like or similar elements of the incubator sound systems. For the purposes of presenting a brief and clear description of the present invention, the preferred embodiment will be discussed as used for providing audio feedback to infants within an incubator. The figures are intended for representative purposes only and should not be considered to be limiting in any respect.

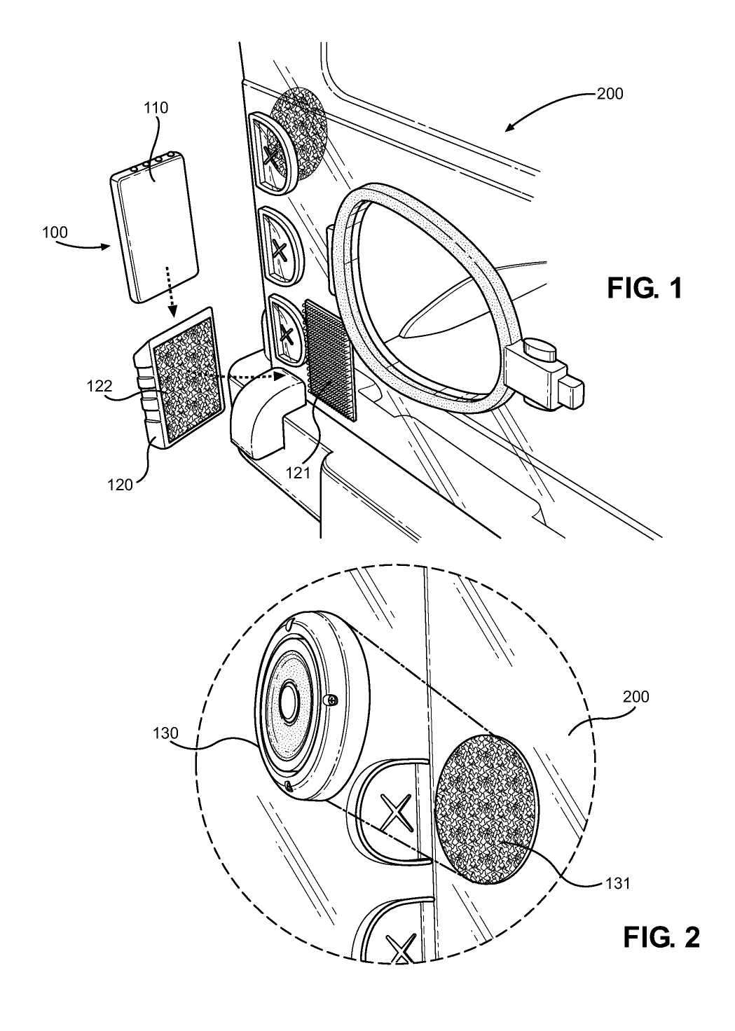

Referring now to FIG. 1, there is shown an incubation unit 200 as is commonly used for warming and monitoring premature infants. A portable sound system 100 is being installed such that audio feedback can be played back within the interior of the incubator device. As is known in the art of infant life support devices, an exemplary incubator is a domed structure having at least one wall with an interior and exterior surface, and defining an interior space. Multiple apertures in the wall(s) are provided to allow the free flow of fresh air through the incubator. Incubators may have temperature regulation systems to increase or decrease internal temperature according to the health needs of a resident infant. Sealable openings may also be provided for enabling caregiver arm access. These larger apertures facilitate interaction between caregivers and incubated infants.

An audio playback device cradle 120 is removably secured to the exterior side of the incubation device wall(s) via a cradle mounting means 121. In the depicted embodiment, the cradle mounting means is a patch of hook and loop fastener secured to the exterior surface of the incubation device wall. A mating patch of hook and loop fastener 122 is disposed along a portion of the cradle. When the two patches are mated, the connection between the hook and loop fasteners maintains the cradle in a mounted position. Other forms of mounting may also be used, such as adhesive pads, buttons, magnetic strips, mounting hooks, and the like.

The audio playback device cradle 120 has at least one sidewall and a bottom, defining an interior volume with an upper opening. A mating mounting means 122 such as hooks, hook and loop fasteners, or adhesive is secured to a portion of the exterior of the cradle sidewall. This mating mounting means engages with the mounting means 121 on the exterior of the incubation device sidewall to form a removable connection capable of supporting the weight of the cradle and the audio playback device. During use, the audio playback device 110 is placed within the interior volume of the cradle for storage. In a preferred embodiment, the walls of the cradle are high enough so as to cower any controls disposed on the audio playback device, thereby reducing the risk that a passerby will bump the controls and modify the audio playback settings.

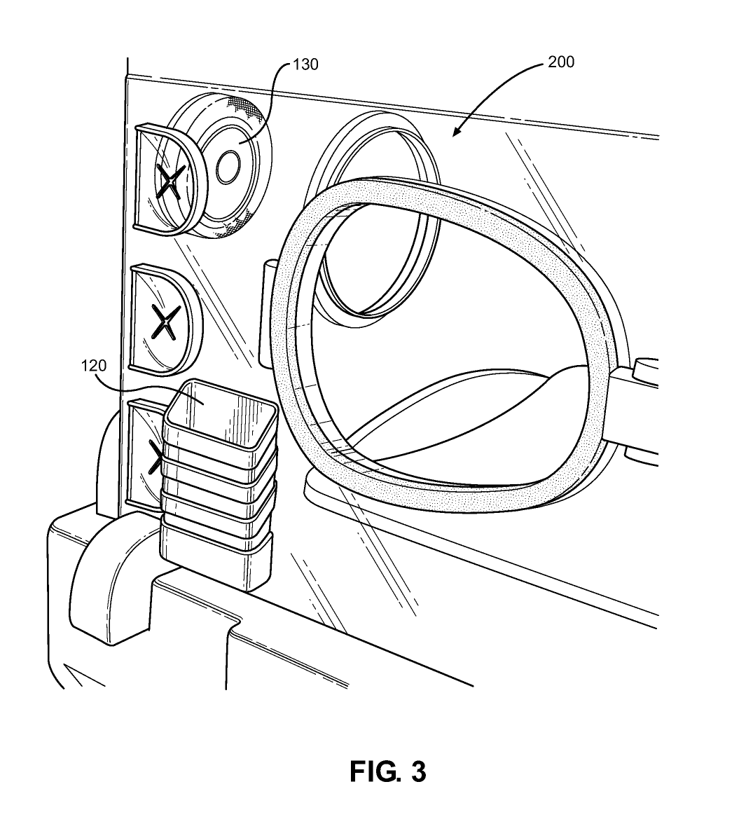

Turning now to FIG. 2, there is displayed an illustration of the speaker being attached to the exterior surface of the incubation device wall. One or more speakers 130 are removably secured to the interior surface of the wall(s) of incubation device 200. The one or more speakers are removably secured to the interior surface of an incubation unit wall by way of a speaker mounting means ∫131. Like the cradle mounting means, the speaker mounting means provides sufficient bond between the speaker and the incubation unit so as to hold the speaker in position during use. In the depicted example the speaker mounting means is hook and loop fasteners, with one half secured to the incubation unit, and the other half (not shown) secured to the back of the speaker unit. Other securing means such as snaps, buttons, adhesive, magnetic strips, etc may be employed as the speaker securing means.

Speakers may come in a variety of shapes and sizes, and the present system is not limited to any particular construction of speaker. Each speaker has an audio input jack that receives an audio jack dongle. An audio output jack is disposed along the casing of the audio out put device, for receiving an opposing end of an audio cable with audio jack dongle. In this way the audio playback device and the speaker(s) are placed in communication, thereby enabling audio signal transfer from the audio playback device to the speaker(s). The removable, modularity of the speakers provides caregivers with an easy way to service or repair malfunctioning speakers.

Referring now to FIG. 3, there is shown an incubation unit with the present system prepped but not in use. The cradle 120 is removably secured to an exterior surface of an incubation unit wall such that the cradle upper opening is directed upwards. The hollow interior of the cradle is empty and ready to receive an audio playback device (not shown). The cradle preferably has solid sides that prevent incidental contact with the audio playback device controls. In this manner, the cradle not only supports the audio playback device but also protects it during use.

A speaker 130 is removably secured to an interior surface of a wall of the incubation unit 200. The speaker should be positioned near the upper portion of the incubation unit, to avoid audio output directly around the ears of an infant. Exposure to excessively high volumes of noise, or prolonged direct exposure to sound waves could cause the infant discomfort, thereby negating the purpose of the present system. As such, placement of the speaker above the position of a sleeping infant is desirous.

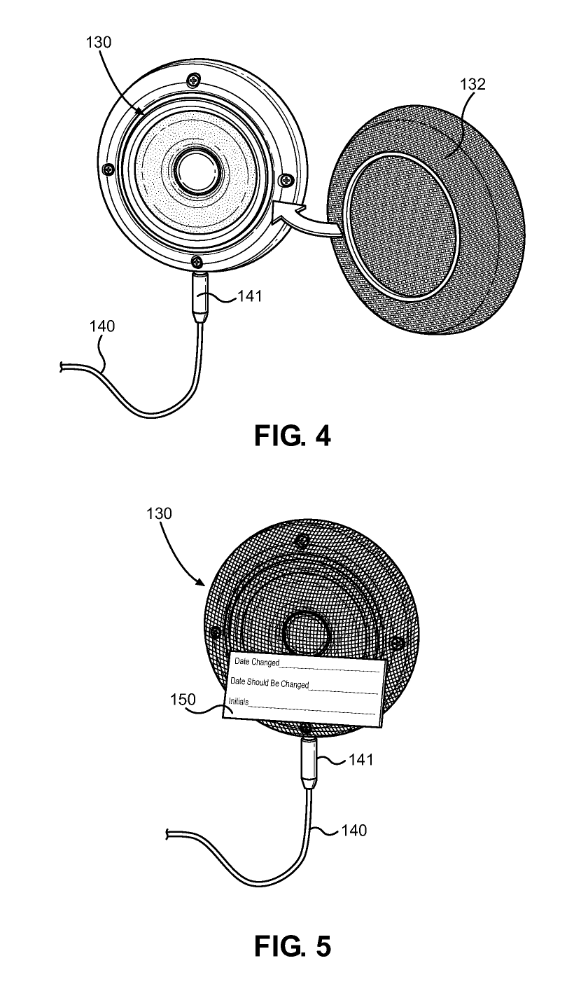

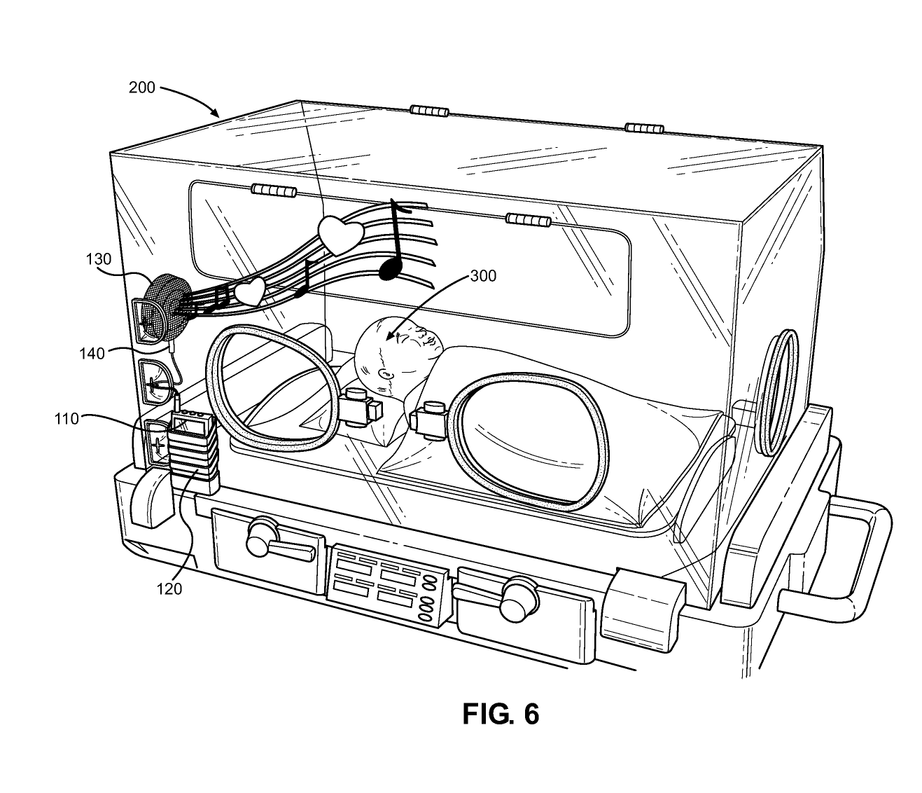

Additional safety features of the present invention are shown in FIGS. 4 and 5. Each speaker unit 130 may be covered with a removable protective shield 132. The shield is a mesh or fabric barrier that reduces the likelihood that germs and bacteria will land on the speaker. In a preferred embodiment the shield is treated with an anti-bacterial or anti-microbial agent to further reduce germ proliferation. Thin fabrics and synthetic materials that permit airflow while filtering out particulate are desirous. By employing protective shields, speakers remain largely sterile throughout use and can quickly be transferred from one incubation unit to another. The in use position of the protective shield does not impede the connection of the audio cable 140 and associated jack dongle 141 to the speaker unit.

Labels 150 are secured to the exterior of the speaker unit 130 when the system is in use. The labels have printed indicia associated with text fields. By way of example, fields for patient name, therapy treatment start date, selected audio playback tracks, and the like, may be prominently displayed on the label. An attending nurse or caregiver writes on the label to fill in these fields, or alternatively fills the fields in on a computer and prints out the resulting label. The label is then affixed to the exterior of the protective shield. Label information aids attendants in ensuring that the proper audio therapy treatment is provided for a pre-determined duration. As will be understood to one of ordinary skill in the art, the label may also be affixed to the exterior surface of the cradle wall(s) or on the incubation unit itself.

Referring to FIG. 6 the audio therapy system is shown in use. A sleeping infant 300 lies within the hollow interior volume of an incubation unit 200. The audio therapy system 100 is installed on the incubation unit. The support cradle 120 is removably mounted along the exterior of an incubation unit wall. An audio playback device 110 is loosely contained within the interior of the cradle. The audio playback device may have indicator lights may be disposed along an upper portion thereof to provide visual indication of whether audio therapy is in progress. One or more speaker units 130 are removably mounted along the interior of an incubation unit wall. Mounting the speakers in this way ensures that sound output is directed towards the interior of the incubation unit, where the incubated infant can perceive it.

An audio cable 140 connects the audio playback device to at least one speaker. As described above, the audio cable may have jack connectors at opposing ends thereof. One jack connects to an audio input port on the speaker(s), while a second jack that connects with an audio output port on the audio playback device. As illustrated in the figure the audio jack cable may be run through an air hole aperture in the incubation unit to effectively place the speaker on the incubation unit interior and the audio playback device on the incubation unit exterior in communication.

The audio playback device may be any portable, reprogrammable device known in the art, such as an mp3 player. The device should have a memory, a processor device, a storage media, an audio output port, a power source, and an input means. The input means may be an alphanumeric keypad, may be a touchscreen interface, or the like. The storage media contains stored programming instructions that when executed, enable the device to receive input from the input means and queue an associated file in storage for audio layback. The programming instructions also enable recordation and storage of audio files. Preferred audio includes the sound of a mother's heartbeat, a voice recording of the mother, soothing music, or nature sounds. Optionally, the audio playback device may have a data transfer port to enable data transfer between the audio playback device and a terminal computer. This functionality aids caregivers by enabling them to transfer audio files to the device from another computer. In another embodiment, the audio playback device may have a wireless transceiver in communication with the processing device. Audio files can be wirelessly transferred to and from the audio playback device from other computers on a shared network.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An audio therapy system for calming incubated infants, comprising:
an audio playback device configured to communicate audio file data;
one or more speaker units, each adapted to receive audio output from said audio playback device;
a cradle having at least one side wall and a bottom, said at least one sidewall having an interior and exterior surface, and wherein said at least one sidewall and said bottom define a hollow interior volume sized to receive and retain said audio playback device;
a cradle mounting means, which removably secures said support cradle to an exterior surface of an incubation unit when in use;
a speaker mounting means, which removably secures said one or more speakers to an interior surface of an incubation unit when in use.

2. The system of claim 1, further comprising:
an audio cable having jacks at opposing ends for connecting said one or more speakers to said audio playback device.

3. The system of claim 1, wherein said audio playback device is an mp3 player.

4. The system of claim 1, further comprising:
at least one protective shield configured to fit over said one or more speaker units, and adapted to filter air.

5. The system of claim 4, wherein said at least one protective shield is treated with an antibacterial or antimicrobial substance.

6. The system of claim 1, further comprising:
a plurality of labels bearing indicia associated with text input.

7. The system of claim 6, wherein said labels have adhesive backing.

8. An audio therapy system for calming incubated infants, comprising:
an audio playback device configured to communicate audio file data;
one or more speaker units, each adapted to receive audio output from said audio playback device;
an audio cable having jacks at opposing ends for connecting said one or more speakers to said audio playback device
a cradle having at least one side wall and a bottom, said at least one sidewall having an interior and exterior surface, and wherein said at least one sidewall and said bottom define a hollow interior volume sized to receive and retain said audio playback device;
a cradle mounting means, which removably secures said support cradle to an exterior surface of an incubation unit when in use;
a speaker mounting means, which removably secures said one or more speakers to an interior surface of an incubation unit when in use;
at least one protective shield configured to fit over said one or more speaker units, and adapted to filter air;
a plurality of labels bearing indicia associated with text input.
